(19) United States
(12) Patent Application Publication
(10) Pub. No.: US 2021/0327580 A1
(43) Pub. Date: Oct. 21, 2021

(54) Method for Stratifying IBS Patients

(71) Applicant: 4D Pharma PLC, Leeds (GB)

(72) Inventors: Fergus SHANAHAN, Kinsale (IE); Paul W. O'TOOLE, Cork (IE); Ian B. JEFFERY, Cork (IE)

(21) Appl. No.: 17/112,433

(22) Filed: Dec. 4, 2020

Related U.S. Application Data
(63) Continuation of application No. PCT/EP2019/065035, filed on Jun. 7, 2019.

(30) Foreign Application Priority Data
Jun. 7, 2018 (EP) 18176641.1

(51) Int. Cl. G16H 50/20; G16H 10/60; G16H 50/70; G16H 70/60; G16H 10/40; G06N 20/20; G06N 5/00; G16B 40/00

(52) U.S. Cl. CPC G16H 50/20; G16H 10/60; G16H 50/70; G16B 40/00; G16H 10/40; G06N 20/20; G06N 5/003; G16H 70/60

(57) ABSTRACT

A computer-implemented method for stratifying a patient with irritable bowel syndrome (IBS). The method comprises detecting the presence, absence, or abundance of multiple bacteria in a biological sample obtained from the patient to generate a patient microbiome profile; and operating a trained classifier on the patient microbiome profile to output a signal stratifying the patient with irritable bowel syndrome (IBS) into a first group or a second group. Stratification of the patient into the first group is indicative that the patient has a not significantly altered microbiome in comparison to the average microbiome not indicative of IBS. Stratification of the patient into the second group is indicative that the patient has an altered microbiome in comparison to the average microbiome not indicative of IBS.

### RELATED APPLICATIONS

This application is a continuation of International Application No. PCT/EP2019/065035, filed Jun. 7, 2019, which claims the benefit of European Application No. 18176641.1, filed Jun. 7, 2018, all of which are hereby incorporated by reference in their entirety.

### TECHNICAL FIELD

This disclosure relates to a system and a method for stratifying irritable bowel syndrome (IBS) patients, and a system and a method for generating a trained classifier for stratifying IBS patients.

### BACKGROUND

IBS is a life-long gastrointestinal disorder, beginning usually in adolescence or early adulthood, and is poorly understood. The effective treatment of IBS represents an unmet need. Available treatments are remedies of limited efficacy, typically of specific symptoms, not cures, and there is a long history of failed drug trials. Moreover, there is low regulatory tolerance for toxicity of remedies in IBS and increasing interest in safe non-traditional drug strategies, such as the manipulation of the microbiome with live biotherapeutics (LBTs).

Irritable bowel syndrome (IBS) is a chronic, debilitating, functional gastrointestinal disorder with estimated population prevalence in Europe between 10 and 15%. It places a significant burden on health resources, with IBS affecting nearly 12% of patients seeking care in primary practice and representing the largest subgroup of patients in gastroenterology clinics. IBS is characterised by abdominal pain or discomfort in association with alteration in either stool form or frequency. These symptoms can be debilitating and lead to a significant reduction in quality of life particularly in the more severely affected. The exact pathophysiology of IBS has not been fully elucidated. However, alterations in the function and composition of the gut microbiota are increasingly being implicated as potential causative or exacerbating factors. One of the strongest indicators for this concept is the elevated risk of developing IBS after an episode of acute infectious gastroenteritis. Prospective studies have demonstrated that up to one third of enteric infections lead to new, persistent IBS symptoms.

Several lines of evidence point to disturbances of host-microbe interactions in at least a subset of patients. Because of the heterogeneity of IBS, there is a need for diagnostic markers by which subsets of patients may be identified to inform more appropriate treatment strategies and enhance the design or interpretation of future therapeutic trials of LBTs thereby increasing the likelihood of successfully achieving an effective alleviation of symptoms.

Inadequacies in their clinical utility have been identified in the so-called clinical subtypes of IBS sufferers based solely on patient-reported symptoms such as constipation, diarrhoea or alterations of symptoms, and how these symptoms are interpreted by the clinician (as discussed in The language of medicine: words as servants and scoundrels. Quigley, E. M., Shanahan, F., (2009) ‘Bad language in gastroenterology’. Clin. Med. 2009:9:2 131-135).

Previous studies of the microbiota composition of patients with IBS indicate that some patients with a normal-like microbiota (i.e. a microbiota composition similar to the microbiota composition of a person without IBS, but dissimilar to the microbiota of a patient with IBS) displayed higher scores for anxiety and depression. Patients with a normal-like microbiota may also be described as having a microbiota composition that is dissimilar to other IBS patients, or a microbiota composition that is dissimilar to IBS patients that have a microbiota that is dissimilar to that of a person without IBS. On the other hand, other patients with IBS with an altered/dysbiotic microbiota (i.e. a microbiota dissimilar to the microbiota of a person without IBS, but similar to the microbiota of a patient with IBS) had on average normal scores for anxiety and depression (see Jeffery I B, O'Toole P W, Ohman L, Claesson M J, Deane J, Quigley E M, Simren M. 2012. “An irritable bowel syndrome subtype defined by species-specific alterations in faecal microbiota.” Gut 61:997-1006). Therefore, studies suggest that patients with IBS should be stratified into two groups: (i) those patients with a gastrointestinal disorder characterised by an altered microbiota and (ii) those patients with a gastrointestinal disorder, but with a normal (or ‘healthy-like’) microbiota. These groups of patients would benefit from different treatment plans, so an alternative approach to the current clinical subtyping should result in more appropriate treatment strategies and better outcomes for patients.

In light of the above, there exists a need for a method that stratifies patients with IBS into two categories: patients with an “altered” microbiota (i.e. group (i) patients) and patients with a “normal-like” microbiota (i.e. group (ii) patients). Conventional computer-implemented methods and systems are not capable of categorising patients into an IBS sub-group with a normal-like microbiome in a reliable and accurate manner. Thus, there exists a need for a computer-implemented method and system that is able to achieve this reliability and accuracy in identifying IBS in this specific group of patients.

US 2017/0270270 A1 relates to a method and a system for microbiome-derived diagnostics and therapeutics in the field of microbiology. The method can classify individuals according to their microbiome composition, including classifying an individual as someone who has IBS upon detection of certain features derived from the microbiome composition. Absent from US 2017/0270270 A1 is disclosure of a method of stratifying patients with IBS into two groups. Individuals can be classified as either having, or not having, IBS (among many other diagnoses) according to their microbiome. Patients with IBS are not stratified into any additional groups at all, let alone groups of patients with ‘altered’ and ‘normal-like’ microbiome profiles.

Also discussed in US 2017/0270270 A1 is testing the efficacy of microbiome composition in predicting characterisations of the patients, i.e. the efficacy of microbiome composition for diagnosis. Certain features of the microbiome can then be identified as having high correlation with a certain diagnosis (IBS, for example). This classifies individuals as either having, or not having, IBS and does not classify IBS patients into two sub-groups.

WO 2014/188378 A1 relates to a method for aiding in the diagnosis of IBS in an individual. The method classifies samples as either IBS samples or non-IBS samples. Like the method of US 2017/0270270 A1, the IBS samples are not classified into sub-groups according to ‘altered’ or ‘normal-like’ microbiome profiles.

In light of the above, there remains a need for a method that stratifies patients with IBS into two categories: patients with an “altered” microbiota (i.e. group (i) patients) and patients with a “normal-like” microbiota (i.e. group (ii) patients).

### SUMMARY

In one aspect, a computer-implemented method for stratifying a patient with IBS into a category based on the microbiome of the patient is provided. The method comprises:
detecting the presence, absence, or abundance of multiple bacteria in a biological sample obtained from the patient to generate a patient microbiome profile; and
operating a trained classifier on the patient microbiome profile to output a signal stratifying the patient with irritable bowel syndrome (IBS) into a first group or a second group;

wherein stratification of the patient into the first group is indicative that the patient has an altered microbiome in comparison to a microbiome not indicative of IBS; and

wherein the stratification of the patient into the second group is indicative that the patient has a not significantly altered microbiome in comparison to a microbiome not indicative of IBS.

Previously, it has been a challenge to accurately stratify patients with IBS that have a “healthy” microbiome and patients with IBS that have an “altered” microbiome from a group of patients. In other words, there is a need for patients with IBS to be categorised into two groups: (i) patients with IBS having an altered microbiome in comparison to the average (i.e. typical or general) microbiome of a patient not having IBS, and (ii) patients with IBS having a not significantly altered microbiome in comparison to the average (i.e. typical or general) microbiome of a person without IBS. Subjects falling outside of groups (i) and (ii) may be described as not having IBS, or as “healthy” individuals. In some examples, these healthy individuals can be identified using the Rome IV Diagnostic Questionnaire, as an optional initial step.

The patients in group (i) may be described as having a microbiome (or “patient microbiome profile”) that is dissimilar to, not the same as, altered, or substantially different to the microbiome of a person without IBS (i.e. a “healthy” individual). In other words, the patients with IBS in group (i) may be described as having an abnormal microbiome in comparison to people without IBS. For instance, the difference between the microbiome profile of a patient in group (i) and the microbiome profile of a “healthy” individual may be above a predetermined threshold. It is also possible that some people with true dysbiosis may be asymptomatic.

The patients in group (ii) may be described as having a microbiome, (or “patient microbiome profile”) that is similar to, the same as, or substantially the same as the microbiome of a person without IBS (i.e. a “healthy” individual). In other words, the patients with IBS in group (ii) may be described as having a ‘healthy’, normal, normal-like or near-normal, microbiome. For instance, the difference between the microbiome profile of a patient in group (ii) and the average microbiome of a “healthy” person may be below a predetermined threshold.

The normal-like microbiome of the patients with IBS in group (ii) may be described as being more similar to the average (i.e. general or typical), microbiome of a healthy person than the microbiome of the altered-microbiome patients in group (i). The microbiome, or the microbiome profile, of patients in group (ii) may be referred to as being “eubiotic-like”. On the other hand, the microbiome, or the microbiome profiles, of patients in group (i) may be referred as being “dysbiotic”.

It is a challenge to accurately identify the normal-like microbiome patients with IBS. However, it has been found that it is possible to classify these patients in an accurate manner by operating a trained classifier on the microbiome profile of such patients. This provides the ability to identify these IBS patients, even when their microbiome is difficult to distinguish from the microbiome of a patient without IBS using conventional means. This can assist in reducing the number of missed, or incorrect, diagnoses that in turn can assist in providing the correct treatment plan for a patient with IBS in order to alleviate their symptoms.

The trained classifier is able to distinguish between patients with IBS in group (i) and those in group (ii) for which different treatments plans may be appropriate. Treating patients with IBS depending on whether they fall in group (i) or group (ii) can lead to more effective outcomes.

In another aspect, a computer-implemented method for generating a trained classifier for stratifying a patient with IBS into a category based on the microbiome of the patient is provided. The method comprises:
obtaining a plurality of microbiome profiles each corresponding to a biological sample;

wherein a first subset of the plurality of microbiome profiles is classified as being indicative of the presence of IBS based on the microbiome data of each microbiome profile in the first subset;

wherein a second subset of the plurality of microbiome profiles is classified as being indicative of the absence of IBS based on the microbiome data of each microbiome profile in the second subset; and
using the microbiome profile of the first subset and the second subset to generate a trained classifier to stratify a patient with irritable bowel syndrome (IBS) into a first group or a second group;

wherein stratification of the patient into the first group is indicative that the patient has an altered microbiome in comparison to the average microbiome not indicative of IBS; and

wherein the stratification of the patient into the second group is indicative that the patient has a not significantly altered microbiome in comparison to the average microbiome not indicative of IBS.

It has been found that by using microbiome profiles that are classified as either being indicative of the presence of IBS or being indicative of the absence of IBS to generate a trained classifier, allows the resulting trained classifier to accurately identify a patient with IBS that has a not significantly altered microbiome in comparison to the average microbiome of a healthy person without IBS. It has been found that the features set out below assist in improving the accuracy of the trained classifier in identifying these patients.

Preferably, the method comprises identifying the first subset and the second subset of the plurality of microbiome profiles based on microbiome data of each one of the microbiome profiles; classifying each microbiome profile of the first subset as being indicative of the presence of IBS; and classifying each microbiome profile of the second subset as being indicative of the absence of IBS.

Preferably, identifying the first subset and the second subset comprises: performing principal component analysis or principal co-ordinate analysis (or another ordination technique) on the microbiome profiles to generate a plurality of data points each corresponding to one of the plurality of microbiome profiles; and identifying the first subset and the second subset based on a spearman correlation dissimilarity metric (or other dissimilarity or distance metrics) between each one of the plurality of data points.

Preferably, using the microbiome profile of the first and second subsets to generate the trained classifier comprises using a feature selection algorithm to identify a plurality of features from the first subset and the second subset; and generating the trained classifier using the plurality of features identified.

Preferably, only the features identified by the feature selection algorithm are used to generate the trained classifier.

Preferably, the feature selection algorithm comprises a regression analysis method.

Preferably, the regression analysis method comprises a least absolute shrinkage and selection operator (LASSO) method, or an elastic net algorithm, or another feature selection methodology.

Preferably, generating the trained classifier using the plurality of features identified comprises generating a predictive model using the random forest machine learning classifier using the plurality of features identified.

Preferably, the random decision forest comprises around 1500 decision trees.

For the LASSO method (or the elastic net algorithm) the lambda parameter, and for the random forest the number of trees is optimised to enhance sensitivity and specificity. The optimisation of these parameters generally depends on the size and type of the dataset, and optimisation is performed using a grid search on the input dataset. The LASSO and random forest algorithm in combination with one another was found to provide good predictive performance.

Preferably, the regression analysis is performed using cross validation.

Preferably, the trained classifier is generated using the plurality of features identified by cross validation.

Preferably, the cross validation is k-fold cross validation.

Preferably, the cross validation is 10-fold cross validation. Using 10-fold cross validation for both the LASSO and random forest algorithms avoids overfitting the models.

Preferably, the 10-fold cross validation is performed without nesting and/or is repeated 10 times.

Preferably, the plurality of microbiome profiles is pre-processed to exclude operational taxonomic units (OTUs) occurring in less than 5% of the microbiome profiles thereby generating a filtered set of microbiome features upon which the trained classier is generated.

In another aspect, a computer-implemented method for stratifying a patient with IBS into a category based on the microbiome of the patient is provided. The method comprises:
obtaining a plurality of microbiome profiles each corresponding to a biological sample;

wherein a first subset of the plurality of microbiome profiles is classified as being indicative of the presence of IBS based on the microbiome data of each microbiome profile in the first subset;

wherein a second subset of the plurality of microbiome profiles is classified as being indicative of the absence of IBS based on the microbiome data of each microbiome profile in the second subset;
using the microbiome profile of the first subset and the second subset to generate a trained classifier to determine the presence or absence of IBS;
detecting the presence, absence, or abundance of multiple bacteria in a biological sample obtained from the patient to generate a patient microbiome profile; and
operating the trained classifier on the patient microbiome profile to stratify a patient with irritable bowel syndrome (IBS) into a first group or a second group;

wherein stratification of the patient into the first group is indicative that the patient has an altered microbiome in comparison to the average microbiome not indicative of IBS; and

wherein the stratification of the patient into the second group is indicative that the patient has a not significantly altered microbiome in comparison to the average microbiome not indicative of IBS.

In one aspect, a computer-implemented method for diagnosing irritable bowel syndrome (IBS) in a patient is provided. The method comprises:
detecting the presence, absence, or abundance of multiple bacteria in a biological sample obtained from the patient to generate a patient microbiome profile; and
operating a trained classifier on the patient microbiome profile to output a signal indicating the presence or absence of IBS in the patient.

In another aspect, a computer-implemented method for stratifying a patient with IBS into a category based on the microbiome of the patient is provided. The method comprises:

detecting the presence, absence, or abundance of multiple bacteria in a biological sample obtained from the patient to generate a patient microbiome profile;

generating a trained classifier based on a training data set comprising a plurality of microbiome profiles by:
using a least absolute shrinkage and selection operator (LASSO) method to select features: and
using the selected features to train a random decision forest;

operating the trained classifier on the patient microbiome profile to output a signal indicating that the patient has: a not significantly altered microbiome in comparison to the average microbiome not indicative of IBS or an altered microbiome in comparison to the average microbiome not indicative of IBS.

In another aspect, there is provided a (e.g. non-transitory) computer-readable medium comprising instructions which, when executed by a computer, cause the computer to carry out one or more of the methods described herein.

In another aspect, there is provided a system comprising a processor and a memory, the memory comprising instructions that, when executed by the processor, cause the processor to perform one or more of the methods described herein.

In another aspect, there is provided a (e.g. non-transitory) data carrier signal carrying the computer program described herein.

### BRIEF DESCRIPTION OF THE DRAWINGS

Embodiments of the invention will be described, by way of example, with reference to the following drawings, in which:

FIG. 1 illustrates a method for generating a trained classifier for stratifying IBS patients;

FIG. 2 illustrates microbiome profiles transformed into a principal co-ordinate analysis ordination;

FIG. 3 illustrates a method for generating the trained classifier in further detail;

FIG. 4 illustrates a method for stratifying IBS patients;

FIG. 5 illustrates results of using the trained classifier to identify IBS patients having a not significantly altered microbiome in comparison to the average microbiome not associated with IBS;

FIG. 6 illustrates results of using the trained classifier to diagnose IBS in patients having an altered microbiome in comparison to the average microbiome not associated with IBS; and

FIG. 7 illustrates a schematic diagram of a system and an electronic device for performing one or more of the methods described herein.

### DETAILED DESCRIPTION

Described herein are methods and systems that are capable of accurately stratifying IBS patients from their microbiome, particularly in cases where a patient's microbiome is similar to the average microbiome of a person without IBS. Previously, it has been a challenge to distinguish this specific sub-group of patients with IBS from those patients with an altered microbiome.

In addition, diagnosis of IBS from a patient's microbiome can lead to a more informed diagnosis than diagnosing IBS from symptoms reported by a patient alone where the latter can lead to variable and inaccurate results and inappropriate treatment strategies. Thus, it is advantageous to be able to also diagnose IBS in patients from their microbiome. In addition, methods and systems are described herein that can be used to generate a trained classifier for performing the diagnosis of IBS. The trained classifier can be stored, for execution by a processor using the microbiome data of a test sample in order to provide an output that indicates the presence or absence of IBS in a patient in an accurate manner.

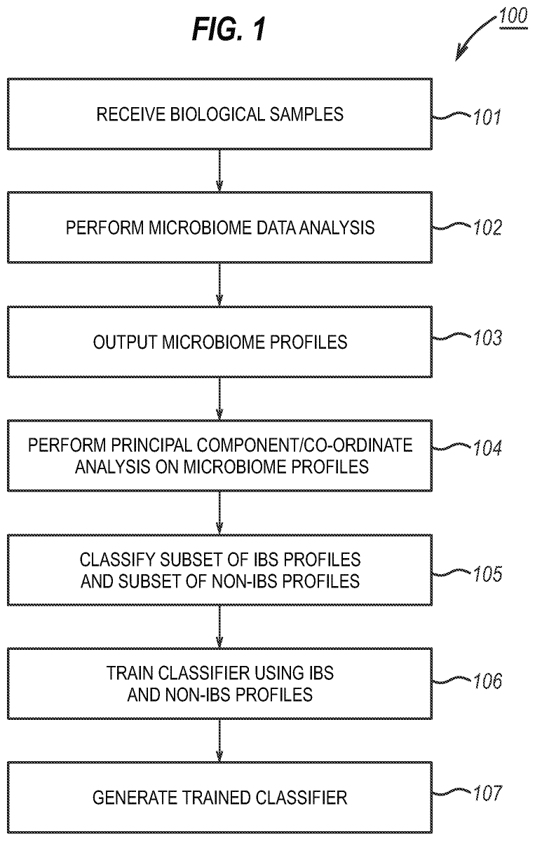

Referring to FIG. 1, there is provided a computer-implemented method 100 for generating a trained classifier for identifying an IBS patient having a not significantly altered microbiome in comparison to the average microbiome not associated with IBS.

In step 101 a plurality of biological samples is obtained, each from a respective patient. Each one of the biological samples can be obtained using a sampling kit. A specific example of a method for obtaining biological samples using a sampling kit is described in greater detail below.

In step 102 microbiome data analysis is performed on each one of the biological samples, and in step 103 a microbiome profile is output for each sample. Each respective microbiome profile indicates the presence, absence, or abundance of multiple bacteria in the biological sample. A specific example of a method for performing the microbiome data analysis and outputting the microbiome profile is described in greater detail below.

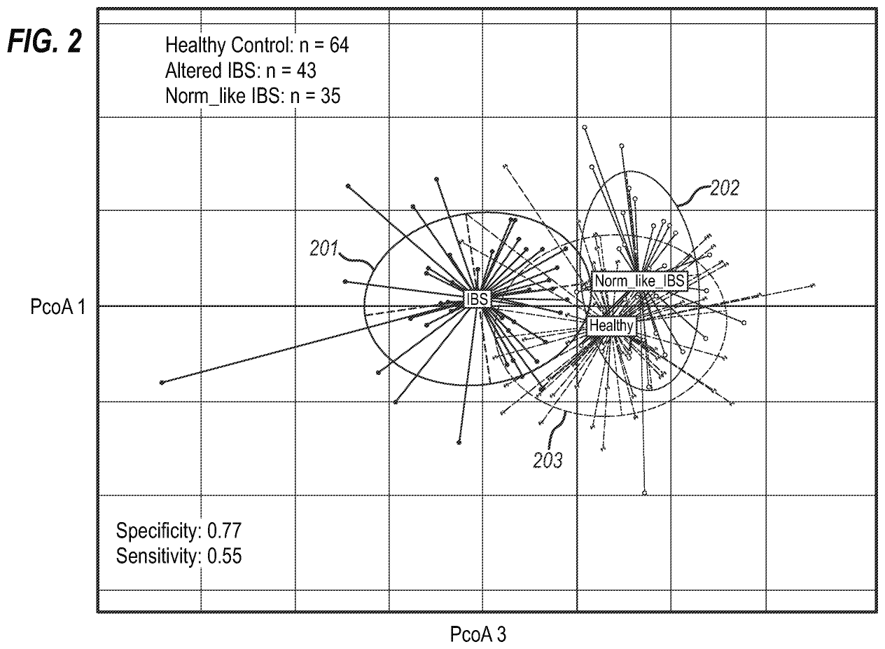

In step 104 principal component analysis (PCA) principal co-ordinate analysis (PCoA), or another ordination technique is performed on the microbiome profiles in order to transform the microbiome profiles into a principal component analysis co-ordinate system. FIG. 2 shows an example of the microbiome profiles transformed into a principal component analysis or principle co-ordinate analysis or other ordination system.

PCA or PCoA is used as the ordination technique to identify trends (eigenvectors) in the microbiome. These trends are summaries of how the taxa abundance changes across the sample space. Once these trends are identified, the trends can be filtered based on their ability to distinguish between healthy patients and those with IBS using linear regression and a P-value of 0.05. This process identified two eigenvectors, the first explaining most of the variance. This eigenvector was used for the rest of the analysis. The second eigenvector identified explains less variance.

With reference to FIG. 2, it can be seen that microbiome profiles 201 that indicate the presence of IBS in a patient are clustered together separately from the microbiome profiles 203 that indicate the absence of IBS (i.e. the “healthy” individuals without IBS). Also, it can be seen that the microbiome profiles 202 of patients with IBS that have a microbiome similar to the healthy patients (i.e. the Norm_like IBS patients) are clustered closely with the microbiome profiles 203 of the healthy individuals. FIG. 2 shows that the cluster of microbiome profiles 202 of the normal-like microbiome IBS patients at least partially overlaps with the cluster of microbiome profiles 203 of the healthy individuals. Therefore, it is difficult to identify the normal-like microbiota IBS subgroup from the healthy individuals from their respective microbiome using principal component analysis or principal co-ordinate analysis alone.

Referring to FIG. 2, separation along the primary axis highlighted a significant separation between the healthy control samples and the IBS cohort and so was used to identify an optimal threshold using ROC (receiver-operator curve) analysis, the optimal threshold providing maximum sensitivity and specificity. This provided an initial stratification of the IBS samples into altered and normal-like microbiome IBS sub-groups based on the optimal threshold of maximal sensitivity and specificity (Youden's J metric). This stratification is shown in FIG. 2.

In step 105 a first subset of the plurality of the microbiome profiles is classified as being indicative of the presence of IBS, and a second subset of the plurality of microbiome profiles is classified as being indicative of the absence of IBS. The first subset and the second subset of microbiome profiles are identified based on the spearman distance between the data points of each microbiome profile in the principal component analysis co-ordinate system. Thus, PcoA or PCA and the spearman dissimilarity metric is the ordination technique used to identify the major trends in the dataset. Other ordination techniques may be used.

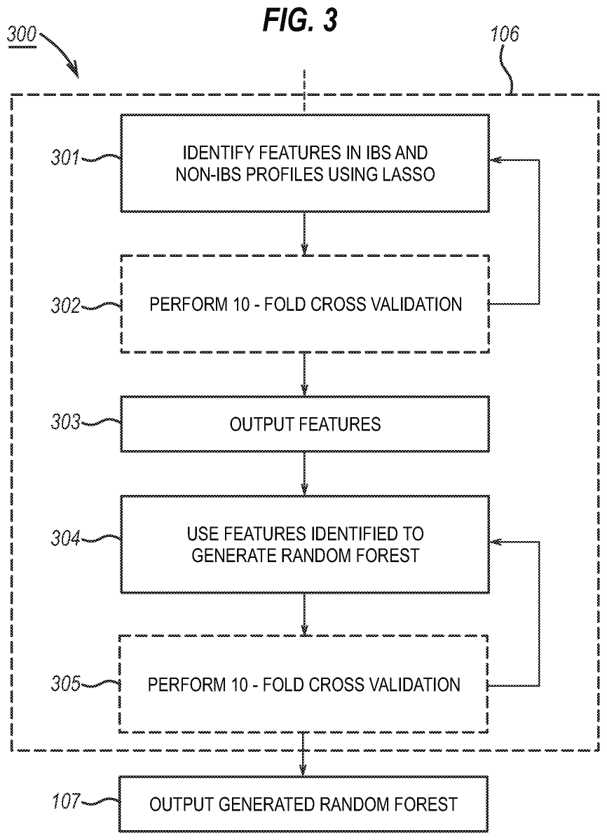

In step 106 the first subset and the second subset of the microbiome profiles are used to train a classifier. In this step the microbiome profiles of only two groups of subjects were used. The first group consists of microbiome profiles of patients with IBS that also have a microbiome that is dissimilar (altered) to the average microbiome of a person without IBS (i.e. group (i) patients). The second group consists of microbiome profiles of “healthy” individuals without IBS. The microbiome profiles of patients with IBS that also have a microbiome that is similar to the average microbiome profiles of “healthy” individuals without IBS (group ii) were not used to train the classifier. The method for training the classifier will be described in greater detail with reference to FIG. 3.

The microbiome profiles used to train the classifier may be pre-processed in order to filter a selection of the microbiome profiles, such that a selection of profiles are not used to train the classifier. For example, the plurality of microbiome profiles can be pre-processed to exclude operational taxonomic units (OTUs) occurring in less than 5% of the microbiome profiles thereby generating a filtered set of microbiome profiles upon which the trained classier is generated. Since microbiome profiles may vary in geographically distinct locations, the features may be optimised based on the population of a geographic location.

In this example, the training data consisted of 64 samples from “healthy” individuals without IBS and samples from the 43 patients from group (i).

In step 107, once the classifier has been trained using the first and second subsets, the trained classifier may be described as having been generated. Once generated, the trained classifier is stored in a data storage resource, such as memory, for later use on test data.

Referring to FIG. 3, there is provided a computer-implemented method 300 for generating the trained classifier for stratifying IBS patients, which is a specific example of step 106 described above.

In step 301 a least absolute shrinkage and selection operator (LASSO) method is used to identify features from the first subset and the second subset of the microbiome profiles identified in step 105. In this example, the LASSO algorithm is used to improve accuracy and interpretability of models by efficiently selecting features. However, an alternative feature selection process could be used instead. This may be a supervised or an unsupervised feature selection process.

In alternative examples, nonparametric approaches to the feature selection process may be used. For instance, the Wilcox Test, Kruskal-Wallis Test, or Mann-Whitney Test could be used. Parametric approaches to the feature selection process may be used, such as linear regression, t-statistic or mixed models. Structured analysis pipelines may be used for feature selection, such as Multivariate Association with Linear Models (MaAsLin), Linear discriminant analysis Effect Size (LefSe) or STAMPs. Other approaches and statistical models may be used, such as area under the curve (AUC) analysis from receiver operating characteristic (ROC), pROC analysis, fold change analysis, DESeq, DESeq2, or metagenomeSeq.

LASSO is a supervised feature selection process that selects the predictive features to be used to train the classifier. In this specific example, the samples are first split into training and test sets. As described with reference to step 105, the training sets used are the first and second subsets. The process iterates through each data point in the training set and puts them into the LASSO linear regression model. LASSO is described in more detail in Journal of the Royal Statistical Society, Series B, 58(1), 1996, R. Tibshirani, “Regression Shrinkage and Selection via the Lasso”, pages 267-288.

The feature selection process may be performed using k-fold cross validation, in step 302, in order to optimise the model. In k-fold cross-validation, the training datasets (i.e. the first subset and the second subset) are randomly split up into a number of groups of equal size. The number of groups is equal to ‘k’. Each one of the k groups is selected in turn as a validation group for testing the model, and the remaining groups are used as the training data. This process is repeated k times, and in each repetition of the process each one of the k groups is used exactly once as the validation data. This outputs k results that can be averaged to produce an averaged result. This process leads to more accurate results because all of the k groups are used for both validation and training, but each of the k groups is used only once for validation. In a specific example, 10-fold cross validation is used to perform feature selection which has been found to improve the accuracy of the resulting model. Thus, 90% of the data is used as a training set and 10% is used as a test set. This is repeated ten times in such a way that all samples are in the test set once. Also, the 10-fold cross validation may be repeated 10 times and/or may be performed without nesting. In one example, the features may be identified by optimising the hyperparameter using a grid search.

The data points, which show high correlation with sample labels, i.e. IBS or “healthy” using LASSO, are output in step 303 as features for classifier training in step 304. In other words, the features (or combination of features) selected by the feature selection process that most accurately predict a test sample as being indicative of IBS or as being healthy are output in step 303 as the selected features for training the classifier in step 304.

In step 304 the features identified using the LASSO method are used to generate a random decision forest (or “random forest”). The random forest generated may comprise around, or exactly, 1500 trees. Using this number of trees for the random forest has been found to optimise the accuracy of the trained classifier.

The random forest may also be generated using k-fold cross validation, in step 305, in order to optimise the model. Again, using k-fold cross validation leads to more accurate results because all of the training data, along with the corresponding features identified in step 301, are used for both validation and training, but each of the k groups of the training data are used only once for validation. In a specific example, 10-fold cross validation is used to generate the random forest, which has been found to improve the accuracy of the resulting model and also makes efficient use of processing resources. Also, the 10-fold cross validation may be repeated 10 times and/or may be performed without nesting.

The same features which show high correlation with sample labels are selected in the same order in the test set to predict the class labels in the test set. Classifier performance can be checked by comparing the predicted class labels with the actual class labels. This feature selection can be applied to the training set to avoid over-fitting and yields similar results to the prediction based on the normally-distributed features alone.

Other classifiers and machine-learning algorithms may be used to analyse the selected features to determine the presence or absence of IBS and/or classify the biological sample into a subset of IBS. For example, support vector machine (SVMs), Kmeans clustering, I Bayes, Naive Bayes, Gradient Tree Boosting, Neural Networks between Class Analysis, Redundancy Analysis, Linear Discriminate Analysis and blending of these different methodologies may alternatively be used to classify the sample or to stratify disease populations. However, random forests have been found to provide enhanced accuracy in identifying patients with IBS when their microbiome is similar to that of a healthy patient.

The above method may be carried out without cross validation. Alternatively, “leave-one-out” cross validation or cross validation based on bootstrapping the dataset may be used.

In step 107 of FIG. 3, which is a specific example of the same step described with reference to FIG. 1, the random forest is generated and stored for use in stratifying IBS patients. This is a specific example of the trained classifier referred to above. Once the trained classifier has been generated, the selected data points—also referred to as features—are used for classification of samples using the trained classifier in order to indicate the presence or absence of IBS, or to identify a sub-population of IBS based on the microbiome.

In the method described with reference to FIG. 3, the method is implemented in R software, and the glmnet package was used for LASSO. Glmnet fits a generalized linear model via penalized maximum likelihood. The regularization path is computed for the LASSO method (or elastic net penalty algorithm) as a grid of values for the regularization parameter lambda (A). The algorithm is extremely fast, and can exploit sparsity in the input matrix X. The predictions can be made from the fitted models.

Glmnet implements logistic regression when the response is categorical. If there are two possible outcomes (e.g. IBS, healthy), the binomial distribution is used, if not the multinomial distribution is used.

For the binomial model, suppose the response variable takes value in G={1,2}. The model can be written in the following form:

$$log \frac{P r ( G = 2 \vert X = x )}{P r ( G = 1 \vert X = x )} = {β}_{0} + {β}^{T} x$$

which is the so-called “logistic” or log-odds transformation.

The objective function for the penalized logistic regression uses the negative binomial log-likelihood, and is:

$${min}_{( {β}_{0} , β ) ϵ {ℝ}^{ρ + 1}} - [ \frac{1}{N} {\sum }_{i = 1}^{N} {y}_{i} \cdot ( {β}_{0} + {x}_{i}^{T} β ) - log ( 1 + {e}^{( {β}_{0} + {x}_{i}^{T} β )} ) ] + λ [ ( 1 - α ) {‖ β ‖}_{2}^{2} / 2 + α {‖ β ‖}_{1} ]$$

over a grid of values of A covering the entire range. The elastic-net penalty is controlled by α, and bridges the gap between lasso (α=1, the default) and ridge (α=0). The tuning parameter A controls the overall strength of the penalty.

Logistic regression is often plagued with degeneracies when p>N, where p is the number of features and N is the number of samples, and exhibits wild behaviour even when N is close to p. The elastic-net penalty alleviates these issues, and regularizes and selects variables as well.

For the optimisation of λ, the glmnet algorithm uses cyclical coordinate descent, which successively optimizes the objective function over each parameter with others fixed, and cycles repeatedly until convergence. The algorithm uses a quadratic approximation to the log-likelihood, and then coordinate descent on the resulting penalized weighted least-squares problem. These constitute an outer and inner loop. The steps for the optimization are described in Jerome Friedman, Trevor Hastie and Rob Tibshirani “Regularization Paths for Generalized Linear Models via Coordinate Descent” Journal of Statistical Software, Vol. 33(1), 1-22 Feb. 2010, specifically section 3 Regularized Logistic Regression, equations (15) through (18).

The randomForest package was used to generate the random forest models. The parameter “ntree” denotes the number of trees in the forest, which should be in principle as large as possible so that each potential model feature has enough opportunities to be selected. The default value is ntree=500 in the package randomForest. The parameter “mtry” denotes the number of features randomly selected as model features at each split. A low value increases the chance of selection of features with small effects, which may contribute to improved prediction performance in cases where they would otherwise be masked by features with large effects. A high value of mtry reduces the risk of having only non-informative candidate features. In the package randomForest, the default value is √p for classification, where p is the number of features of the dataset. The parameter “nodesize” represents the minimum size of terminal nodes. Setting this number larger causes smaller trees to grow. The default value is 1 for classification. Boulesteix, Anne-Laure et al. “Overview of random forest methodology and practical guidance with emphasis on computational biology and bioinformatics” (2012) provides more detailed descriptions of the parameters within the random forest algorithm.

The machine leaning pipeline described above uses the grid search technique to optimize the parameters (e.g. ntrees). In the grid search several models were generated using different number of trees (e.g. ntrees=500, 1000, 1500, 2000), with different mtry values (e.g. mtry=1, 2, 3, 4, 5, 6, 7, 8, 9, 10). The nodesize parameter was kept at 1, the value for classification. Sensitivity and specificity performance metric was then used to choose the best model, with the optimized mtry and number of trees parameters. In this example, the optimum number of trees was found to be 1500.

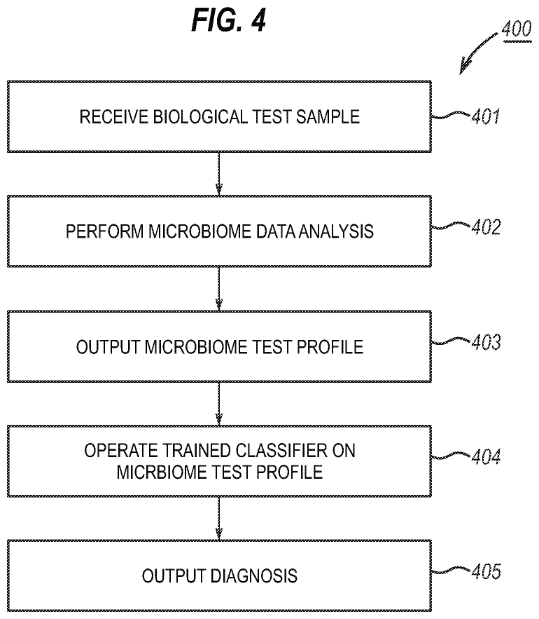

Referring to FIG. 4, there is provided a computer-implemented method 400 for identifying a patient with IBS as having a not significantly altered microbiome (i.e. a “normal-like” microbiome) in comparison to the average microbiome not associated with IBS.

In step 401 a biological test sample is obtained from a patient in a similar manner to that described with reference to step 101, which is discussed in greater detail below.

In step 402 microbiome data analysis is performed on the biological test samples, and in step 403 a microbiome data test profile is output for the test sample. The microbiome data test profile indicates the presence, absence, or abundance of multiple bacteria in the biological test sample. Steps 402 and 403 are carried out in a similar manner to that described with reference to steps 102 and 103 which are discussed in greater detail below.

In step 404 the microbiome data test profile is input to the trained classifier generated as described with reference to FIGS. 1 to 3. In this step the classifier is operated on the microbiome test profile and outputs a signal identifying the patient as a group (i) patient or a group (ii) patient. In another example, the trained classifier is operated on the microbiome data test profile and outputs a signal indicative of the presence or absence of IBS in the patient corresponding with the microbiome data test profile.

The trained classifier may output a probability of the presence or absence of IBS, such as a probability between 0 and 1. If this probability meets a predetermined threshold probability, this may output an indication of the presence of IBS, or in another example stratification of the patient into group (i). On the other hand, if this probability does not meet a predetermined threshold probability, this may output an indication of the absence of IBS or in another example stratification of the patients into group (ii). The probability may be configurable so that the output can be tuned for accuracy. In one example, the probability is 50%, or 0.5. Thus, if the probability output is 0.5 or below, this indicates the absence of IBS (or that the individual is “healthy), and if the probability output is above 0.5, this indicates an individual with IBS.

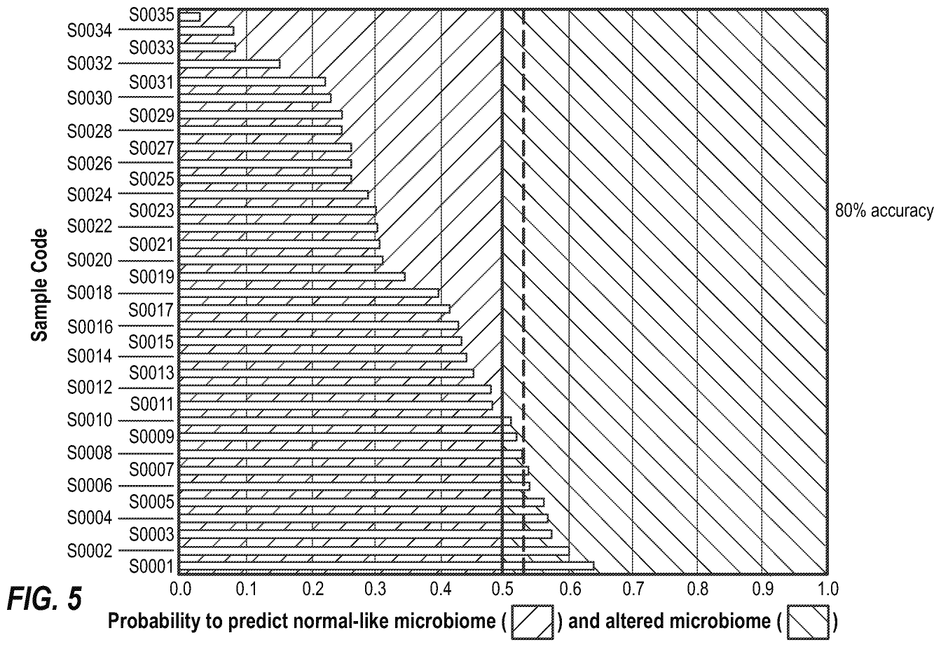

The trained classifier was found to be able to diagnose IBS in patients having a microbiome similar to the average microbiome of a patient without IBS (i.e. group (ii) patients that have a “normal-like” microbiome). The accuracy of the trained classifier to diagnose these patients was found to be around 80%. This is illustrated in FIG. 5, in which 35 samples of group (ii) patients are shown. The samples below the optimised threshold represented by the dotted line are classified as group (ii) samples, while the samples above the threshold are classified as group (i) samples. The optimised threshold is between 0.5 and 0.6, and in this specific examples the threshold is 0.53, although the threshold can be tuned to a different value.

Of the 35 samples, 28 were correctly classified as being indicative of the presence of IBS and a microbiome substantially the same as the microbiome of a person without IBS (i.e. a microbiome of a group (ii) IBS patient). In addition, only 7 out of 35 samples were misclassified as being indicative of a microbiome substantially different to the microbiome of a person without IBS (i.e. a microbiome of a group (i) IBS patient).

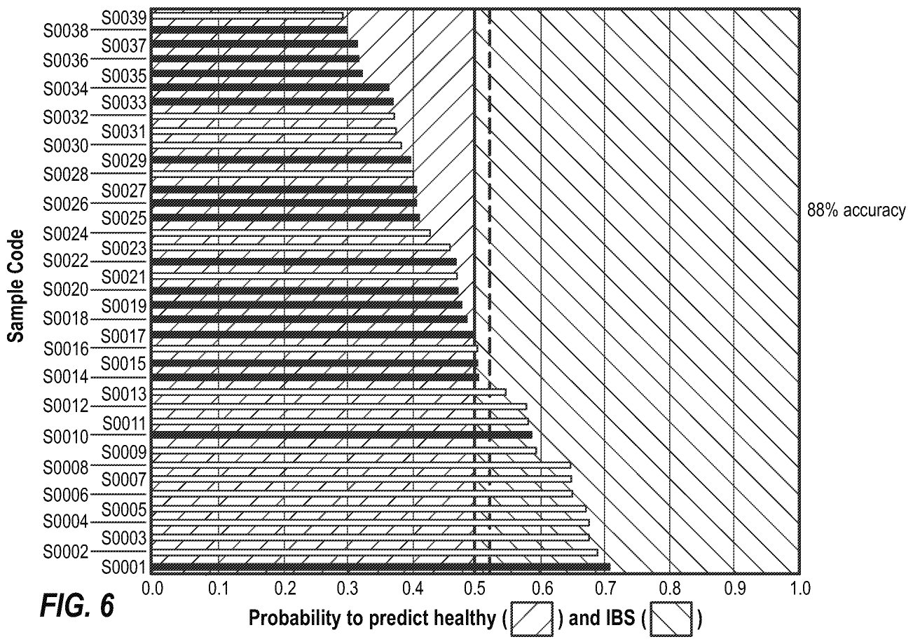

In addition, the trained classifier was found to be able to diagnose IBS in patients having a microbiome dissimilar to the average microbiome of a person without IBS, and the trained classifier was found to be able to diagnose individuals as not having IBS. The accuracy of the trained classifier to diagnose these individuals was found to be around 88%. This is illustrated in FIG. 6, which shows only 39 out of a total of 107 test samples. The black bars designate “healthy” individuals, and the white bars designate patients with IBS. As shown in FIG. 6, only 5 healthy samples were misclassified as having IBS (i.e. samples S0001, S0010, S0014, S0015 and S0017), and only 8 IBS samples were misclassified as being “healthy” (i.e. samples S0039, S0032, S0031, S0030, S0028, S0024, S0023 and S0021). Therefore, only 13 samples from 107 samples were misclassified giving an accuracy of −88%.

One example of obtaining the biological samples referred to in steps 101 and 401 may involve using the “DNeasy Blood & Tissue Kit” from Qiagen of 19300 Germantown Road, Germantown, Md. 20874 USA to obtain the biological samples. This kit is used to extract microbial DNA from 0.2 g of each of 145 frozen faecal samples obtained from patients.

16S rRNA gene amplicons preparation and sequencing is performed on the obtained samples using the 16S Sequencing Library Preparation Nextera protocol developed by Illumina 5200 of Illumine Way, San Diego, Calif. 92122 USA. In this process, 50 ng of each of the DNA faecal extracts is amplified using PCR and primers targeting the V3-V4 variable region of the 16S rRNA gene. The products are purified, and forward and reverse barcodes are attached by a second round of adapter PCR. The resulting PCR products are purified, quantified and equimolar amounts of each amplicon were then pooled before being sent for sequencing.

One example of performing the microbiome data analysis to output the microbiome profiles, as referred to in steps 102, 103, 402 and 403, involves first sequencing the biological samples to generate raw amplicon sequence data. Then, the returned raw amplicon sequence data are merged and trimmed using the well-known flash methodology. This generates a single read from the read pairs and also filters out low quality reads that do not contain sequence similarity in the overlapping region. The USEARCH pipeline methodology (version 8.1.1861_i86_linux64) is used to identify singletons and hide them from the OTU (Operational Taxonomic Unit) generating step. This is done to reduce the complexity of the data and improve the overall quality due to the likelihood of these reads being low quality and therefore generating low quality OTUs. The reads are retained within the overall analysis by their reintroduction in the final mapping step.

The UPARSE algorithm is used to cluster the sequences into OTUs. This generates a list of sequences which are likely to reflect the true taxonomic variation. Due to the generation of chimeric sequences during the wet-lab amplification step of the generation of the 16S dataset, the UCHIME chimera removal algorithm was used with the Chimeraslayer reference database to remove chimeric sequences. Chimeric sequences occur when two sequences combine to generate a new sequence due to annealing of the 16S sequences which share a high-level of similarity, even when the origin of these sequences are from phylogenetically distinct origins. Then, the USEARCH global alignment algorithm is used to map all reads, including singletons onto the remaining OTU sequences. Scripts are used to generate the OTU abundance information using the read assignment as classified by the USEARCH global alignment algorithm. This grouping of sequences into OTUs generates microbiome compositional information, in terms of abundance and diversity. These steps allow the abundance of each taxa associated sequence in each sample to be estimated. In addition, as the raw sequences are mapped to the OTU sequences generated from only high-quality data, there can be a high-level of confidence that the raw sequences are mapped to sequences of biological origin.

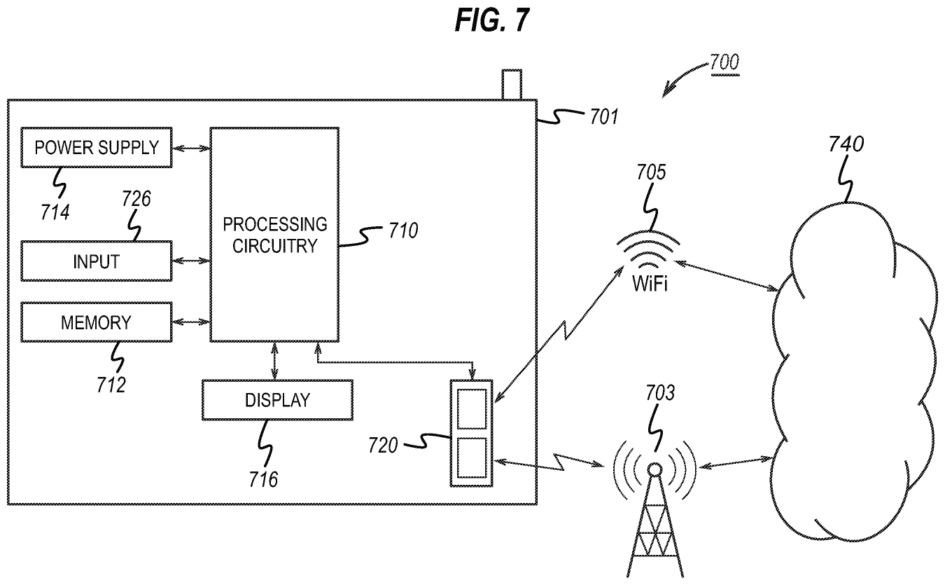

FIG. 7 shows a system 700 comprising an exemplary electronic device 701 configured to perform one or more of the methods described herein. The electronic device 701 comprises processing circuitry 710 (such as a microprocessor) and a memory 712. The electronic device 701 also comprises one or more of the following subsystems: a power supply 714, a display 716, a transceiver 720, and an input 726.

Processing circuitry 710 may control the operation of the electronic device 701 and the connected subsystems to which the processing circuitry is communicatively coupled. Memory 712 may comprise one or more of random access memory (RAM), read only memory (ROM), non-volatile random access memory (NVRAM), flash memory, other volatile memory, and other non-volatile memory.

Display 716 may be communicatively coupled with the processing circuitry 710, which may be configured to cause the display 716 to output images indicating the diagnosis, or data relating to the diagnosis, determined by one or more of the methods described herein.

The display 716 may comprise a touch sensitive interface, such as a touch screen display. The display 716 may be used to interact with software that runs on the processor 710 of the electronic device 701. The touch sensitive interface permits a user to provide input to the processing circuitry 710 via a discreet touch, touches, or one or more gestures for controlling the operation of the processing circuitry and the functions described herein. It will be appreciated that other forms of input interface may additionally or alternatively be employed for the same purpose, such as the input 726 which may comprise a keyboard or a mouse at the input device. The input 726 and/or the display 716 may be configured to input the microbiome profiles used to train the classifier, or to input the microbiome test profile used to output a diagnosis. The microbiome profile and/or the microbiome data test profiles may be received at the electronic device 701 via the transceiver 720.

The transceiver 720 may be one or more long-range RF transceivers that are configured to operate according to communication standard such as LTE, UMTS, 3G, EDGE, GPRS, GSM, and Wi-Fi. For example, electronic device 701 may comprise a cellular transceiver that is configured to communicate with a cell tower 703 via a cellular data protocol such as LTE, UMTS, 3G, EDGE, GPRS, or GS. The electronic device 701 may comprise a Wi-Fi transceiver that is configured to communicate with a wireless access point 705 via a Wi-Fi standard such as 802.11 ac/n/g/b/a.

Electronic device 701 may be configured to communicate via the transceiver 720 with a network 740. Network 740 may be a wide area network, such as the Internet, or a local area network. Electronic device 701 may be further configured to communicate via the transceiver 720 and network 740 with one or more systems or devices. For instance, the microbiome profile and/or the microbiome data test profiles may be received at the electronic device 701 from one or more system or devices in the network 740 via the transceiver 720.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

Those skilled in the art will realise that storage devices utilised to store program instructions can be distributed across a network. For example, a remote computer may store an example of the process described as software. A local computer may access the remote computer and download a part or all of the software to run the program. Alternatively, the local computer may download pieces of the software as needed, or execute some software instructions at the local terminal and some at the remote computer (or computer network). Those skilled in the art will also realise that by utilizing conventional techniques known to those skilled in the art that all, or a portion of the software instructions may be carried out by a dedicated circuit, such as a DSP, programmable logic array, or the like.

The steps of the methods described herein may be carried out in any suitable order, or simultaneously where appropriate. Additionally, individual blocks may be deleted from any of the methods. Aspects of any of the examples described above may be combined with aspects of any of the other examples described to form further examples without losing the effect sought.

### List of Numbered Embodiments

1. A computer-implemented method for generating a trained classifier for stratifying a patient with irritable bowel syndrome (IBS), the method comprising:

obtaining a plurality of microbiome profiles each corresponding to a biological sample;
wherein a first subset of the plurality of microbiome profiles is classified as being indicative of the presence of IBS based on the microbiome data of each microbiome profile in the first subset;
wherein a second subset of the plurality of microbiome profiles is classified as being indicative of the absence of IBS based on the microbiome data of each microbiome profile in the second subset; and

using the microbiome profiles of the first subset and the second subset to generate a trained classifier to stratify a patient with irritable bowel syndrome (IBS) into a first group or a second group;

wherein stratification of the patient into the first group is indicative that the patient has an altered microbiome in comparison to the average microbiome not indicative of IBS; and

wherein the stratification of the patient into the second group is indicative that the patient has a not significantly altered microbiome in comparison to the average microbiome not indicative of IBS.

2. The computer-implemented method of embodiment 1 comprising:

identifying the first subset and the second subset of the plurality of microbiome profiles based on microbiome data of each one of the microbiome profiles;

classifying each microbiome profile of the first subset as being indicative of the presence of IBS; and

classifying each microbiome profile of the second subset as being indicative of the absence of IBS.

3. The computer-implemented method of embodiment 2 wherein identifying the first subset and the second subset comprises:

performing principal component analysis or principal co-ordinate analysis on the microbiome profiles to generate a plurality of data points each corresponding to one of the plurality of microbiome profiles; and

identifying the first subset and the second subset based on a spearman distance between each one of the plurality of data points.

4. The computer-implemented method of any one of the preceding embodiments wherein using the microbiome profile of the first and second subsets to generate the trained classifier comprises:

using a feature selection algorithm to identify a plurality of features from the first subset and the second subset; and

generating the trained classifier using the plurality of features identified.

5. The computer-implemented method of embodiment 4 wherein only the features identified by the feature selection algorithm are used to generate the trained classifier.
6. The computer-implemented method of embodiment 4 or embodiment 5 wherein the feature selection algorithm comprises a regression analysis method.
7. The computer-implemented method of embodiment 6 wherein the regression analysis method comprises a least absolute shrinkage and selection operator (LASSO) method.
8. The computer-implemented method of embodiment 6 or 7 wherein the regression analysis method is performed using cross validation.
9. The computer-implemented method of embodiment 8 wherein the cross validation is k-fold cross validation.
10. The computer-implemented method of embodiment 8 or embodiment 9 wherein the cross validation is 10-fold cross validation.
11. The computer-implemented method of embodiment 10 wherein the 10-fold cross validation is repeated 10 times.
12. The computer-implemented invention of any one of embodiments 8-11 wherein cross validation is performed without nesting.
13. The computer-implemented method of any one of embodiments 4-12 wherein generating the trained classifier using the plurality of features identified comprises:

generating a random decision forest using the plurality of features identified.

14. The computer-implemented method of embodiment 13 wherein the random decision forest comprises around 1500 decision trees.
15. The computer-implemented method of embodiment 4 to 14 wherein the trained classifier is generated using the plurality of features identified by cross validation.
16. The computer-implemented method of embodiment 15 wherein the cross validation is k-fold cross validation.
17. The computer-implemented method of embodiment 15 or 16 wherein the cross validation is 10-fold cross validation.
18. The computer-implemented method of embodiment 17 wherein the 10-fold cross validation is repeated 10 times.
19. The computer-implemented invention according to any one of embodiments 15-18 wherein cross validation is performed without nesting.
20. The computer-implemented method of any one of the preceding embodiments wherein the trained classifier is arranged to diagnose the presence or absence of irritable bowel syndrome (IBS) in an individual having a not significantly altered microbiome in comparison to the average microbiome not indicative of IBS.
21. The computer-implemented method of any one of the preceding embodiments wherein the plurality of microbiome profiles are pre-processed to exclude operational taxonomic units (OTUs) occurring in less than 5% of the microbiome profiles thereby generating a filtered set of microbiome profiles upon which the trained classier is generated.
22. The computer-implemented method of any one of the preceding embodiments wherein only the microbiome profiles of the first subset and the second subset to generate a trained classifier to determine the presence or absence of IBS in a patient.
23. The computer-implemented method of any one of the preceding embodiments wherein microbiome profiles of patients having a not significantly altered microbiome in comparison to the average microbiome not indicative of IBS are not used as training data to generate the trained classifier.
24. The computer-implemented method of embodiment 23 wherein the microbiome profiles of patients having a not significantly altered microbiome in comparison to the average microbiome not indicative of IBS are used as validation data only for the trained classifier.
25. A computer-implemented method for stratifying a patient with irritable bowel syndrome (IBS), the method comprising:

detecting the presence, absence, or abundance of multiple bacteria in a biological sample obtained from the patient to generate a patient microbiome profile; and

operating a trained classifier on the patient microbiome profile to output a signal stratifying a patient with irritable bowel syndrome (IBS) into a first group or a second group;

wherein stratification of the patient into the first group is indicative that the patient has an altered microbiome in comparison to the average microbiome not indicative of IBS;

wherein the stratification of the patient into the second group is indicative that the patient has a not significantly altered microbiome in comparison to the average microbiome not indicative of IBS;

wherein the trained classifier is generated according to the computer-implemented method of any one of the preceding embodiments.

26. A computer-implemented method for stratifying a patient with irritable bowel syndrome (IBS), the method comprising:

detecting the presence, absence, or abundance of multiple bacteria in a biological sample obtained from the patient to generate a patient microbiome profile;

generating a trained classifier based on a training data set comprising a plurality of microbiome profiles by:
using a least absolute shrinkage and selection operator (LASSO) method to select features: and
using the selected features to train a random decision forest;

operating the trained classifier on the patient microbiome profile to output a signal stratifying a patient with irritable bowel syndrome (IBS) into a first group or a second group;

wherein stratification of the patient into the first group is indicative that the patient has an altered microbiome in comparison to the average microbiome not indicative of IBS; and

wherein the stratification of the patient into the second group is indicative that the patient has a not significantly altered microbiome in comparison to the average microbiome not indicative of IBS.

27. A computer-implemented method for diagnosing the presence or absence of irritable bowel syndrome (IBS) in a group of patients comprising a patient having a not significantly altered microbiome in comparison to the average microbiome not indicative of IBS, a patient having an altered microbiome and a patient having a microbiome not indicative of IBS, the method comprising:

detecting the presence or absence of multiple bacteria in a biological sample obtained from at least one of the patients to generate a patient microbiome profile; and

operating a trained classifier on the patient microbiome profile to output a signal indicating the presence or absence of IBS in the patient.

28. A computer-readable medium comprising instructions which, when executed by a computer, cause the computer to carry out the method of any preceding embodiment.
29. A system comprising a processor and a memory, the memory comprising instructions that, when executed by the processor, cause the processor to perform the method of any one of embodiments 1 to 28.

1.-15. (canceled)

16. A method for treating a subject with irritable bowel syndrome (IBS) comprising providing to the subject a treatment for IBS based on stratifying the subject by a method comprising:
(a) accessing in computer memory a trained machine learning classifier for stratifying a patient with IBS, wherein the trained machine learning classifier has been trained at least in part by:
(i) obtaining a plurality of microbiome profiles each corresponding to a biological sample;
wherein a first subset of the plurality of microbiome profiles is indicative of a presence of IBS; and
wherein a second subset of the plurality of microbiome profiles is indicative of an absence of IBS; and
(ii) using the microbiome profiles of the first subset and the second subset to generate the trained machine learning classifier for stratifying a subject with IBS into a first group or a second group;
wherein the stratifying of the subject into the first group is indicative that the subject has a significantly altered microbiome in comparison to a reference microbiome not indicative of IBS; and
wherein the stratifying of the subject into the second group is indicative that the subject does not have a significantly altered microbiome in comparison to the reference microbiome not indicative of IBS;
(b) obtaining a test microbiome profile corresponding to a biological sample obtained or derived from the subject with IBS;
(c) processing the test microbiome profile using the trained machine learning classifier to stratify the subject with IBS into the first group or the second group.

17. The method of claim 16, wherein (ii) further comprises:
identifying the first subset and the second subset of the plurality of microbiome profiles based on microbiome data of each one of the microbiome profiles;
classifying each microbiome profile of the first subset as being indicative of the presence of IBS; and
classifying each microbiome profile of the second subset as being indicative of the absence of IBS.

18. The method of claim 17, wherein identifying the first subset and the second subset comprises:
performing principal component analysis or principal co-ordinate analysis on the microbiome profiles to generate a plurality of data points each corresponding to one of the plurality of microbiome profiles; and
identifying the first subset and the second subset based at least in part on a Spearman distance between each one of the plurality of data points.

19. The method of claim 16, wherein (ii) further comprises:
using a feature selection algorithm to identify a plurality of features from the first subset and the second subset; and
generating the trained machine learning classifier using the plurality of features identified.

20. The method of claim 19, wherein only the plurality of features identified by the feature selection algorithm is used to generate the trained machine learning classifier.

21. The method of claim 19, wherein the feature selection algorithm comprises a regression analysis method.

22. The method of claim 21, wherein the regression analysis method comprises a least absolute shrinkage and selection operator (LASSO) method or an elastic net algorithm.

23. The method of claim 21, wherein the regression analysis method is performed using cross validation.

24. The method of claim 19, wherein generating the trained machine learning classifier using the plurality of features identified comprises:
generating a random decision forest using the plurality of features identified.

25. The method of claim 24, wherein the random decision forest comprises about 1500 decision trees.

26. The method of claim 19, wherein the trained machine learning classifier is generated using the plurality of features identified by cross validation.

27. The method of claim 26, wherein the cross validation comprises a k-fold cross validation.

28. The method of claim 26, wherein the cross validation comprises a 10-fold cross validation.

29. The method of claim 28, wherein the 10-fold cross validation is repeated 10 times.

30. The method of claim 16, wherein the trained machine learning classifier is configured to detect a presence or an absence of IBS in a subject having a microbiome that is not significantly altered in comparison to a reference microbiome not indicative of IBS, and/or wherein the plurality of microbiome profiles are pre-processed to exclude operational taxonomic units (OTUs) occurring in less than 5% of the microbiome profiles thereby generating a filtered set of microbiome profiles upon which the trained machine learning classifier is generated.

31. The method of claim 16, wherein only the microbiome profiles of the first subset and the second subset are used to generate the trained machine learning classifier, and/or wherein microbiome profiles of subjects not having a significantly altered microbiome in comparison to the reference microbiome not indicative of IBS are not used as training data to generate the trained machine learning classifier.

32. The method of claim 31, wherein the microbiome profiles of subjects not having a significantly altered microbiome in comparison to the reference microbiome not indicative of IBS are used as validation data only for generating the trained machine learning classifier.

33. A computer-implemented method for stratifying a subject with irritable bowel syndrome (IBS), the method comprising:
(a) obtaining a plurality of sequencing reads generated at least in part by performing 16S sequencing of microbial DNA from a biological sample obtained from the subject;
(b) processing the plurality of sequencing reads using a global alignment algorithm, thereby aligning the plurality of sequencing reads onto a plurality of operational taxonomic unit (OTU) sequences;
(c) determining an abundance of a set of OTUs represented in the microbial DNA, based at least in part on the aligning in (b), thereby generating a microbiome profile of the subject; and
(d) processing the microbiome profile of the subject using a trained machine learning classifier to stratify the subject with IBS into a first group or a second group;
wherein the stratifying of the subject into the first group is indicative that the subject has a significantly altered microbiome in comparison to a reference microbiome not indicative of IBS; and
wherein the stratifying of the subject into the second group is indicative that the subject does not have a significantly altered microbiome in comparison to the reference microbiome not indicative of IBS.

34. The method of claim 33, further comprising, prior to (b), processing the plurality of sequencing reads using a greedy OTU clustering algorithm, thereby clustering the plurality of sequencing reads into a plurality of OTUs.

35. A method for treating a subject with irritable bowel syndrome (IBS), comprising:
(a) obtaining a test microbiome profile corresponding to a biological sample obtained or derived from the subject;
(b) processing the test microbiome profile using a trained machine learning classifier to stratify the subject into a first group indicative of having a significantly altered microbiome in comparison to a reference microbiome not indicative of IBS or a second group indicative of not having a significantly altered microbiome in comparison to the reference microbiome not indicative of IBS;
wherein the trained machine learning classifier is trained at least in part by:
(i) obtaining a plurality of microbiome profiles each corresponding to a biological sample;
wherein a first subset of the plurality of microbiome profiles is indicative of a presence of IBS; and
wherein a second subset of the plurality of microbiome profiles is indicative of an absence of IBS; and
(ii) using the microbiome profiles of the first subset and the second subset to generate the trained machine learning classifier for stratifying a subject with IBS into the first group or the second group; and
(c) providing to the subject a treatment for IBS based on the stratifying in (b).
